(19) United States
(12) Patent Application Publication
(10) Pub. No.: US 2025/0303087 A1
(43) Pub. Date: Oct. 2, 2025

(54) MEDICAL DEVICE, CONTROL METHOD, AND NON-TRANSITORY STORAGE MEDIUM STORING PROGRAM THEREOF

(71) Applicant: FUKUDA DENSHI CO., LTD., Tokyo (JP)

(72) Inventors: Ryotaro HASHIMOTO, Tokyo (JP); Mai KOBAYASHI, Tokyo (JP); Tatsuya YONEYAMA, Tokyo (JP)

(73) Assignee: FUKUDA DENSHI CO., LTD., Tokyo (JP)

(21) Appl. No.: 19/089,016

(22) Filed: Mar. 25, 2025

(30) Foreign Application Priority Data
Mar. 29, 2024 (JP) 2024-056637

(51) Int. Cl. A61M 16/00

(52) U.S. Cl. CPC A61M 16/026; A61M 2016/0039; A61M 2016/0042

(57) ABSTRACT

Provided is a medical device for supplying gas of positive pressure to an airway of a patient. The medical device includes a controller that controls supply pressure, and the controller starts pressure reducing control on the supply pressure of gas when a decrease of a flow rate of the gas by a predetermined value from a peak flow rate in an inspiration period of the patient is detected.

### BACKGROUND

The present disclosure relates to a medical device, a control method, and a non-transitory storage medium storing a program thereof, and particularly relates to a medical device that supplies air to the airway of a patient, a control method, and a non-transitory storage medium storing a program thereof.

An artificial respirator used for non-invasive positive pressure ventilation (NPPV) therapy, a continuous positive airway pressure device (hereinafter referred to also as a CPAP device) used for nasal CPAP therapy, and the like are known as medical devices that supply air to the airway of a patient. CPAP is a treatment method that suppresses the occurrence of apnea or hypopnea by continuously feeding the airway of the patient with air having appropriate pressure through a nasal mask and thus preventing the obstruction of the airway during a sleep.

A flow of the air of positive pressure supplied by these medical devices and a flow of air in an expiration of the patient are in opposite directions from each other. The patient may hence have an uncomfortable feeling when the patient performs a respiration. Accordingly, JP-T-2007-524446 describes a CPAP device that achieves an alleviation of the uncomfortable feeling by raising supply air pressure to treatment pressure at a timing of change in the flow rate of the air from negative to positive and stopping the air supply at a timing of change in the flow rate of the air from positive to negative.

### SUMMARY

The CPAP device described in JP-T-2007-524446 performs a pressure raising operation or stops the air supply on the basis of only a timing of inversion of the sign of the flow rate of the air or a timing of the flow rate becoming a threshold value. It is difficult to exhale when the treatment pressure is maintained until the flow rate changes from positive to negative, for example. There is thus further room for the alleviation of an uncomfortable feeling in a respiration motion of the patient.

In view of such problems of the technology in the related art, the present disclosure in one aspect thereof provides a medical device, a control method, and a non-transitory storage medium storing a program thereof that can improve comfort during at least one of an inspiration and an expiration.

The present disclosure in one aspect thereof provides a medical device for supplying air of positive pressure to an airway of a patient, the medical device including controller for controlling the pressure of the air, the controller being configured to start pressure reducing control on the pressure of the air when a decrease in a flow rate of the air by a predetermined ratio from a peak flow rate in an inspiration period of the patient is detected.

According to the present disclosure, it is possible to provide a medical device and a control method thereof that can improve comfort during at least one of an inspiration and an expiration.

### BRIEF DESCRIPTION OF THE DRAWINGS

FIG. 1 is a block diagram illustrating an example of a functional configuration of a CPAP device according to an embodiment;

FIG. 2 is a diagram of assistance in explaining an example of supply air pressure control by the CPAP device according to the embodiment; and

FIG. 3 is a flowchart of operation of the CPAP device according to the embodiment.

### DETAILED DESCRIPTION OF THE PREFERRED EMBODIMENTS

The present disclosure will hereinafter be described in detail on the basis of an illustrative embodiment of the present disclosure with reference to the accompanying drawings. It is to be noted that the following embodiment does not limit the disclosure according to claims. In addition, while a plurality of features are described in the embodiment, not all of the features are necessarily essential to the disclosure, and the plurality of features may freely be combined with each other. Further, in the accompanying drawings, identical or similar configurations are identified by the same reference numerals, and repeated description thereof will be omitted. In addition, while a mode in which the present disclosure is carried out in a CPAP device will be described in the following, the present disclosure can also be carried out in another medical device (for example, an artificial respirator) that has a function of supplying air to the airway of a patient.

### (Example of Configuration of CPAP Device)

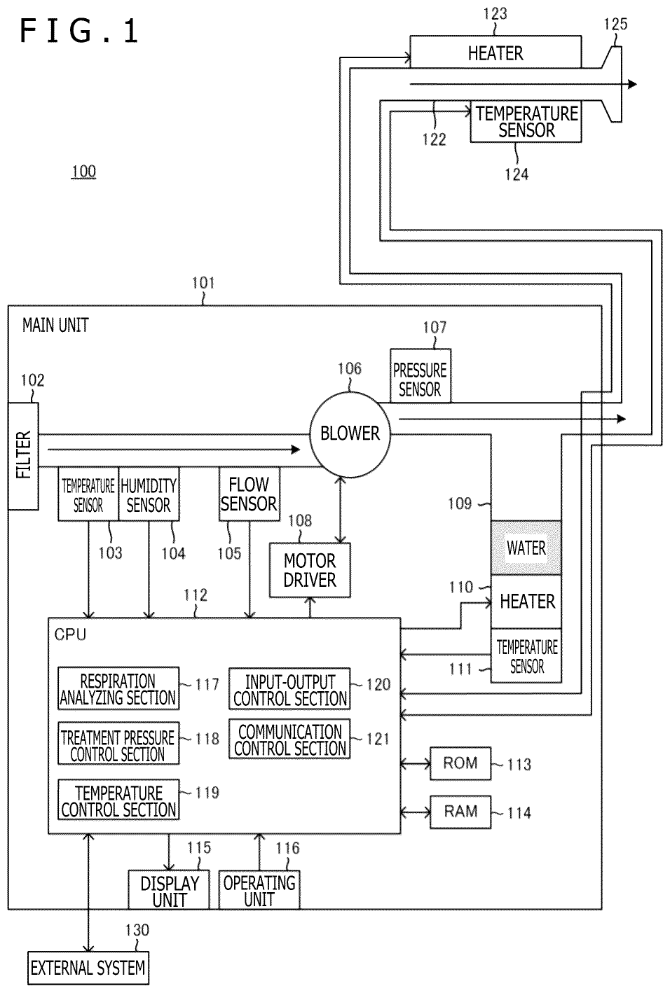

FIG. 1 is a block diagram illustrating an example of a functional configuration of a CPAP device according to one embodiment of the present disclosure.

The CPAP device 100 includes a main unit 101, a mask 125, and a tube 122 that connects the main unit 101 and the mask 125 to each other. The operation of the CPAP device 100 is implemented by a central processing unit (CPU) 112 reading a program stored in a read only memory (ROM) 113 into a random access memory (RAM) 114 and executing the program. Thus, a device including the CPU 112, the ROM 113, and the RAM 114 may be regarded as a computer. Incidentally, functional blocks 117 through 121 provided within the CPU 112 are schematic representations of main functions of various functions that are implemented by the CPU 112 executing the program. Hence, the operation described with the functional blocks 117 through 121 as entities is actually implemented by the CPU 112 executing the program. Instead of this, one or more functional blocks may be implemented by use of a hardware circuit other than the CPU 112.

Constituent elements present on an air flow passage will first be described. A filter 102 is provided at an air intake port to remove pollen, bacteria, dust, and the like. A temperature sensor 103 measures the temperature of air that flows in. The value measured by the temperature sensor 103 is supplied to a temperature control section 119. A humidity sensor 104 measures the humidity of the air that flows in. The value measured by the humidity sensor 104 is supplied to the temperature control section 119. Incidentally, while the supply of air to the patient is assumed in this case, another type of gas such as an oxygen gas or a mixture of air and an oxygen gas may be supplied. In a case where gas other than air is to be supplied to the patient, means (cylinder or the like) for supplying the gas may be connected upstream of the filter 102.

A flow (differential pressure) sensor 105 (hereinafter designated simply as a flow sensor 105) is a differential pressure type flow rate sensor, for example. The flow sensor 105 measures the flow rate of the air within the flow passage on the basis of a pressure difference between an upstream side and a downstream side. Suppose in this case that a positive measured value is obtained when pressure on the upstream side is higher than pressure on the downstream side and that a negative measured value is obtained when the pressure on the downstream side is higher than the pressure on the upstream side. This makes it possible also to identify a flow direction of the air within the flow passage from the measured value of the flow sensor 105. The measured value of the flow sensor 105 is supplied to a respiration analyzing section 117. Incidentally, the flow sensor 105 may measure a flow velocity. The flow rate is Cross-Sectional Area of Flow Passage×Flow Velocity, and the cross-sectional area of the flow passage does not affect the present disclosure. Hence, “the flow rate” in the following description is equivalent to “the flow rate or the flow velocity.”

A blower 106 internally has an impeller and a motor that drives the impeller. A treatment pressure control section 118 controls the rotational speed of the motor through a motor driver 108. The flow rate and supply pressure (supply air pressure) of the air to be supplied to a patient can thereby be adjusted.

A pressure sensor 107 is provided downstream of the blower 106 on the flow passage. The pressure sensor 107 measures the pressure within the flow passage. The measured value of the pressure sensor 107 is supplied to the treatment pressure control section 118. The treatment pressure control section 118 controls the supply air pressure, assuming that the patient is supplied with the air at the pressure measured by the pressure sensor 107.

A humidifier 109 has a water storage tank, and humidifies the air to be supplied to the patient. In this case, the temperature control section 119 controls the temperature of a heater 110 provided to the humidifier 109. An amount of water that would be vaporized from the water storage tank, that is, a degree of humidification, is thereby controlled. A temperature sensor 111 measures the temperature of the heater 110 and supplies the temperature of the heater 110 to the temperature control section 119. In a case where the humidification and the temperature adjustment are performed separately from each other, the heater 110 and the temperature sensor 111 do not have to be provided to the humidifier 109. In addition, air may be blown onto the surface of water within the water storage tank, or the flow passage may be so disposed as to pass through the water, for example.

The tube 122 connects the main unit 101 and the mask 125 to each other. The tube 122 has elasticity and can easily be bent in order to be able to readily follow movement of the mask 125. In addition, the tube 122 is provided with a heater 123 that adjusts the temperature of the air to be supplied to the patient and a temperature sensor 124 that measures the temperature of the air supplied to the patient. The measured value obtained by the temperature sensor 124 is supplied to the temperature control section 119. Incidentally, the tube 122 may adjust the temperature of the air supplied to the patient, by controlling the temperature of water vapor vaporized from a water storage tank by the heater 110 of the humidifier 109, in place of the heater 123 or in addition to the heater 123.

The mask 125 has such a size and shape as to cover at least one of the nose and mouth of the patient. The mask 125 is fitted to the patient by a string or a band that is adjustable in length.

A display unit 115 is, for example, a display provided to a casing of the main unit 101. The display unit 115 displays a message related to the handling of the CPAP device 100, various kinds of menu screens for making settings in the CPAP device 100 and the like, measured values of various kinds of sensors, and the like. The display of the display unit 115 is controlled by an input-output control section 120.

An operating unit 116 is a general term of input devices that can be operated by a user, such as buttons and switches provided to the casing of the main unit 101, for example. In a case where the display unit 115 is a touch display, the display unit 115 and the operating unit 116 are formed integrally with each other. An operation on the operating unit 116 is detected by the input-output control section 120. The CPU 112 performs an action according to the detected operation.

The respiration analyzing section 117 detects various states related to the respiration of the patient and the occurrence of a predetermined event on the basis of the flow rate measured by the flow sensor 105. The event may be, for example, one or more of obstructive apnea, central apnea, hypopnea, flow limitation (partial obstruction of respiration), snore, and Cheyne-Stokes respiration (CRS), but is not limited to these. Incidentally, CRS is a characteristic respiration waveform in which apnea and hyperventilation occur successively. When the respiration analyzing section 117 detects the occurrence of the predetermined event, the respiration analyzing section 117 notifies the treatment pressure control section 118.

The treatment pressure control section 118 controls the operation of the blower 106 such that the supply air pressure becomes a target value, on the basis of the measured value obtained by the pressure sensor 107. In addition, the treatment pressure control section 118 controls the supply air pressure on the basis of information as to whether an expiration is being performed or an inspiration is being performed, the information being detected by the respiration analyzing section 117. Specifically, the treatment pressure control section 118 controls the supply air pressure to treatment pressure when an inspiration is being performed, and controls the supply air pressure to lowest pressure when an expiration is being performed. The treatment pressure control section 118 controls the supply air pressure by providing the motor driver 108 with a duty ratio of a pulsed voltage to be applied to the motor, for example, and thereby controlling the rotational speed of the impeller of the blower 106. The treatment pressure control section 118 notifies the temperature control section 119 of the present supply air pressure.

The temperature control section 119 controls the temperature and humidity of the air to be supplied to the patient, by controlling the operation of the heater 110 while using at least the measured value obtained by the temperature sensor 111. The temperature control section 119 may control the temperature and humidity of the air to be supplied to the patient, by controlling the operation of the heaters 110 and 123 while further taking into consideration one or more of the measured value obtained by the temperature sensor 103, the measured value obtained by the humidity sensor 104, the measured value obtained by the temperature sensor 124, and the supply air pressure notified from the treatment pressure control section 118. Even when the temperature of the heater 110 is constant, a humidifying effect is lower in a case of a high flow rate than in a case of a low flow rate. Hence, the humidity of the air to be supplied to the patient can be controlled more appropriately by controlling the temperature of the heater 110 with the supply air pressure being taken into consideration. The temperature and humidity of the air supplied to the patient may be a temperature and a humidity which the temperature control section 119 has been notified of through the input-output control section 120, that is, a temperature and a humidity set by the user through the operating unit 116.

A communication control section 121 performs processing related to communication between the main unit 101 and an external system 130. The communication control section 121 can perform communication with the external system 130 while complying with one or more of publicly known wireless and/or wire communication standards, for example. The external system 130 may be, for example, a management system for in-hospital medical examination and treatment data or a remote management system for the CPAP device 100.

### (Supply Air Pressure Control Method)

The following problems can occur when a pressure raising operation is performed or the air supply is stopped on the basis of only a timing of inversion of the sign of the flow rate of the air or a timing of the flow rate becoming a threshold value, as described in JP-T-2007-524446.
The air is supplied at the treatment pressure until a timing of the flow rate of the air becoming negative from positive. Thus, exhalation is not easily performed.
The supply air pressure starts to be raised toward the treatment pressure at a timing of the flow rate of the air becoming positive from negative. However, inspiration is difficult to perform when the supply air pressure is raised earlier than a timing of the actual start of the inspiration.

Supply air pressure control in the present embodiment can remedy at least one of such problems.

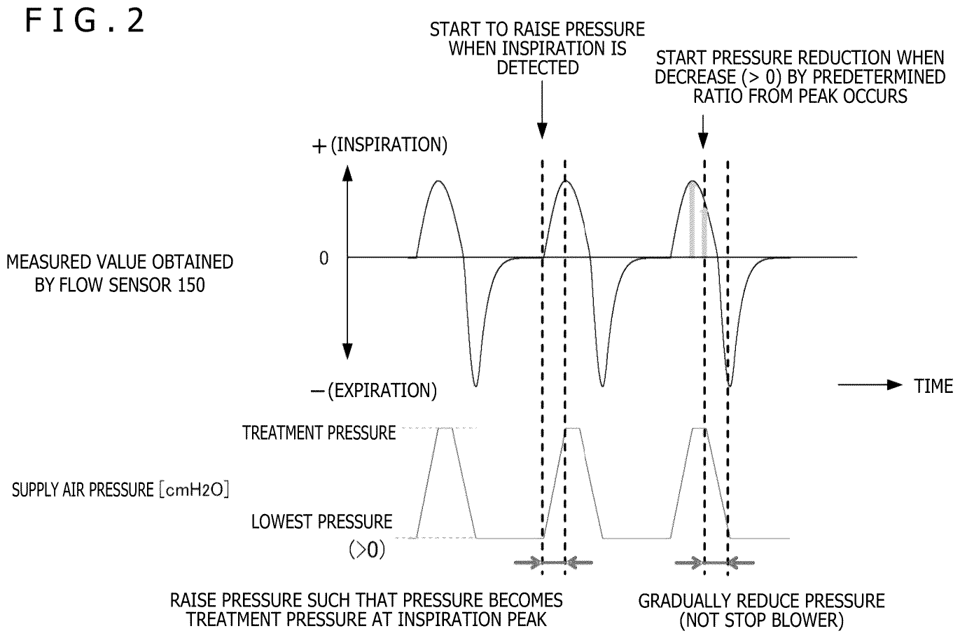

FIG. 2 is a diagram of assistance in explaining an example of the supply air pressure control performed by the CPAP device 100 in the present embodiment. An upper part of FIG. 2 illustrates temporal changes in the measured value obtained by the flow sensor 105 (the flow rate or the flow velocity). In addition, a lower part illustrates temporal changes in the supply air pressure controlled by the treatment pressure control section 118.

As described above, the respiration analyzing section 117 detects various states related to the respiration of the patient on the basis of changes in the value of the flow rate measured by the flow sensor 105. Here, suppose that the respiration analyzing section 117 detects, as the states related to the respiration of the patient, (1) whether an inspiration is being performed or an expiration is being performed, (2) a start timing of the inspiration, and (3) a timing of decrease of the flow rate by a certain ratio from a peak flow rate in an inspiration period. The respiration analyzing section 117 may detect another state related to the respiration of the patient. In addition, the respiration analyzing section 117 calculates a predicted length of time that would be taken for the flow rate to reach a peak from the start of the inspiration.

The respiration analyzing section 117 detects, as the start timing of the inspiration, a time point at which the sign of the measured value obtained by the flow sensor 105 changes from negative to positive, for example. The respiration analyzing section 117 may detect, as the start timing of the inspiration, a time point at which the sign of the measured value obtained by the flow sensor 105 changes from negative to zero.

In addition, the respiration analyzing section 117 calculates the predicted length of time that would be taken for the flow rate to reach a peak from the start of the present inspiration, on the basis of the lengths of time that have been taken for the flow rate to reach a peak from starts of the most recent, predetermined plurality of times of inspiration, for example. As an example, the respiration analyzing section 117 can calculate the predicted length of time on the basis of the lengths of time that have been taken for the flow rate to reach a peak from starts of inspirations in the most recent 1 to 30 times of respiration.

In a case of using a length of time taken for the flow rate to reach a peak from a start of an inspiration in the most recent one time of respiration, the respiration analyzing section 117 uses, as the predicted length of time, the length of time taken for the flow rate to reach a peak from the start of the inspiration in the most recent one time of respiration. However, prediction accuracy may be low in this case. It is hence preferable to calculate, as the predicted length of time, an average of lengths of time taken for the flow rate to reach a peak from starts of inspirations in the most recent two or more times of respiration.

Meanwhile, an upper limit of the number of respirations can be determined as appropriate in consideration of a length of time necessary for obtaining a first value or the like. However, an increase in the number of times decreases a characteristic of following variations in the number of respirations per unit time. Hence, 15 times or less is preferable, and eight times or less is more preferable. Incidentally, in a case where lengths of time taken for the flow rate to reach a peak from starts of inspirations in the most recent n times or more (n≥2) of respiration are used for the calculation of the predicted length of time, the respiration analyzing section 117 may calculate, as the predicted length of time, a weighted average value with the weights of the most recent predetermined number of times m (m<n) increased.

When the respiration analyzing section 117 detects the start timing of an inspiration, the respiration analyzing section 117 notifies the treatment pressure control section 118 of the predicted length of time to be taken to reach the peak flow rate. When notified of the predicted length of time from the respiration analyzing section 117, the treatment pressure control section 118 starts pressure raising control on the supply air pressure. That is, the treatment pressure control section 118 starts control of the blower 106 in such a manner as to raise the supply air pressure at such an increase rate that the supply air pressure reaches a predetermined treatment pressure after the predicted length of time from the reception of the notification from the respiration analyzing section 117 (that is, after the predicted length of time from the start of the inspiration). The increase rate may be fixed or may not be fixed. The treatment pressure may be a fixed value set in advance by a doctor according to the patient, or may be a value automatically set by the treatment pressure control section 118 according to the event detected by the respiration analyzing section 117. Incidentally, suppose that the range of the treatment pressure automatically set by the treatment pressure control section 118 is set in advance by the doctor according to the patient. The treatment pressure or the range of the treatment pressure is set through the operating unit 116 or the external system 130, and is stored in the ROM 113.

In addition, a lower limit value (lowest pressure) of the supply air pressure is similarly set through the operating unit 116 or the external system 130 by the doctor and is stored in the ROM 113. The lowest pressure can be set in a range equal to or higher than 0 cmH₂O but lower than the treatment pressure. Incidentally, the lowest pressure may be set as an absolute value, or may be set as an amount of decrease in pressure (for example, in units of 1 cmH₂O) from the treatment pressure. Incidentally, making the lowest pressure higher than 0 cmH₂O can suppress a decrease in a treatment effect. In addition, making the lowest pressure higher than 0 cmH₂O provides another advantage of being able to raise the supply air pressure smoothly at a time of the pressure raising control.

A sharp change in the supply air pressure can cause an uncomfortable feeling to the patient. The treatment pressure control section 118 hence controls the blower 106 in such a manner as to increase the supply air pressure at a fixed or substantially fixed increase rate both at a time of a pressure increase and at a time of a pressure reduction. As described above, the treatment pressure control section 118 controls the supply air pressure by feedback control using the measured value obtained by the pressure sensor 107.

As described above, in the present embodiment, when a start of an inspiration is detected, the supply air pressure is increased in such a manner as to become the treatment pressure at a peak of the inspiration. The patient hence does not readily experience a feeling of strangeness at a time of the inspiration, and can perform a natural inspiration. In addition, a length of time taken to reach the treatment pressure is dynamically predicted on the basis of the most recent plurality of times of respiration. Therefore, even when the respiration intervals of the patient change, the patient is not readily given a feeling of strangeness.

In addition, the respiration analyzing section 117 determines that an inspiration flow rate has reached a peak when the measured value of the flow sensor 105 changes from an increase to a decrease. Further, the respiration analyzing section 117 updates the predicted length of time by using a length of time taken for the inspiration flow rate to reach a peak from the detection of the start of the inspiration.

Further, the respiration analyzing section 117 detects, as a start timing of pressure reducing control, a timing of decrease of the measured value of the flow sensor 105, that is, the flow rate, by a predetermined value from the peak flow rate. The predetermined value may be a fixed value, or may be a ratio to the peak flow rate. In a case of the ratio to the peak flow rate, the ratio is preferably equal to or more than 25% but equal to or less than 90%, and is more preferably equal to or more than 30% but equal to or less than 50%. FIG. 2 illustrates an example of detecting a timing of decrease of the measured value obtained by the flow sensor 105 by 33% from the peak flow rate (the measured value becoming the peak flow rate*0.67).

When detecting the start timing of the pressure reducing control, the respiration analyzing section 117 notifies the treatment pressure control section 118. The treatment pressure control section 118 reduces the supply air pressure (starts the pressure reducing control on the supply air pressure) in response to this notification. In the pressure reducing control on the supply air pressure, the treatment pressure control section 118 controls the blower 106 such that the supply air pressure decreases at a fixed rate (pressure reducing ratio) determined in advance in the program stored in the ROM 113, instead of stopping the blower 106. The treatment pressure control section 118 stops the pressure reduction when the supply air pressure reaches the lowest pressure determined in advance.

The treatment pressure control section 118 controls the blower 106 such that the supply air pressure decreases at the pressure reducing ratio determined in advance from the pressure reduction start timing. Here, the pressure reducing ratio can be defined as a value at which the lowest pressure is reached in a pressure reduction period of a length determined in advance in the program stored in the ROM 113 from the pressure reduction start timing. The pressure reduction period is preferably a length equal to or more than 400 ms but equal to or less than 1000 ms. When the pressure reduction period is shorter than 400 ms, the pressure decreases quickly, and hence, the treatment effect is decreased. In contrast, when the pressure reduction period is longer than 1000 ms, the pressure decreases slowly, and hence, a difficulty in exhalation tends to be felt.

As described above, in the present embodiment, when it is determined that the inspiration flow rate has reached a peak, the supply air pressure is decreased at the fixed rate (pressure reducing ratio) determined in advance, at the timing of decrease of the flow rate by a predetermined value from the peak flow rate. Hence, a natural pressure reduction synchronized with a change from an inspiration motion to an expiration motion of the patient can be realized, so that the patient is not readily given a feeling of strangeness.

In addition, the supply air pressure is decreased before the sign of the measured value obtained by the flow sensor 105 changes from positive to negative (or the measured value obtained by the flow sensor 105 changes from a positive value to zero) and at a fixed pressure reducing ratio. It is thus possible to alleviate a difficulty in exhalation as compared with control that stops the blower at a time point of change of the sign of the measured value from positive to negative (or change of the measured value from a positive value to zero). In addition, in a case where the lowest pressure is set higher than 0 cmH₂O, it is possible to suppress a decrease in the treatment effect, and raise the supply air pressure smoothly at a time of the pressure raising control.

Incidentally, in a case where the measured value obtained by the flow sensor 105 has a plurality of peaks in the inspiration period (period in which the measured value obtained by the flow sensor 105 is positive), the supply air pressure control that facilitates inspiration can be realized when a first peak is used for the calculation of the predicted length of time. Meanwhile, the pressure reduction start timing is determined by use of a first peak after the predicted length of time.

Only one of the pressure raising control and the pressure reducing control on the supply air pressure as described above may be performed, or both may be performed.

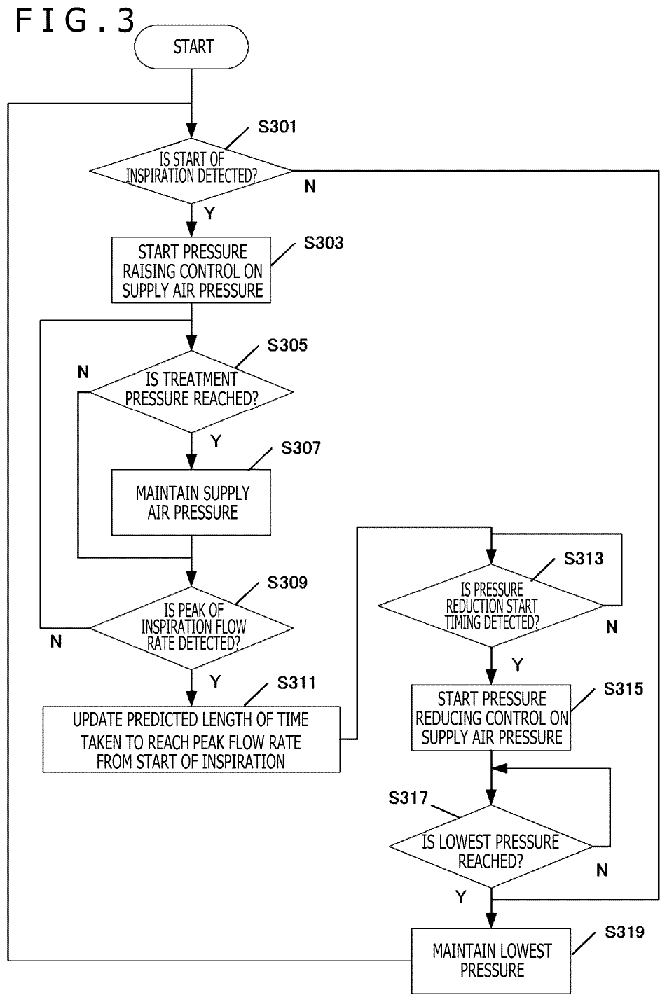

The above-described supply air pressure control operation will be further described with reference to a flowchart illustrated in FIG. 3.

In S301, the respiration analyzing section 117 determines whether an inspiration start timing is detected. When the respiration analyzing section 117 determines that the inspiration start timing is detected, the respiration analyzing section 117 notifies the treatment pressure control section 118 of the predicted length of time taken to reach the peak flow rate, and advances the processing to S303. When the respiration analyzing section 117 does not determine that the inspiration start timing is detected, the respiration analyzing section 117 advances the processing to S319. When the inspiration start timing is not detected, the treatment pressure control section 118 in S319 controls the blower 106 such that the supply air pressure maintains the lowest pressure. The treatment pressure control section 118 then returns the processing to S301.

In S303, the treatment pressure control section 118 starts the pressure raising control on the supply air pressure. As described above, the treatment pressure control section 118 controls the blower 106 in such a manner as to raise the supply air pressure at such an increase rate that the supply air pressure reaches the predetermined treatment pressure after the predicted length of time from the reception of the notification from the respiration analyzing section 117 (that is, after the predicted length of time from the start of the inspiration).

In S305, the treatment pressure control section 118 determines whether the supply air pressure has reached the treatment pressure. When the treatment pressure control section 118 determines that the supply air pressure has reached the treatment pressure, the treatment pressure control section 118 advances the processing to S307. When the treatment pressure control section 118 does not determine that the supply air pressure has reached the treatment pressure, the treatment pressure control section 118 advances the processing to S309.

In S307, the treatment pressure control section 118 ends the pressure raising control on the supply air pressure, and continues the control of the blower 106 in such a manner as to maintain the supply air pressure at the treatment pressure.

In S309, the respiration analyzing section 117 determines whether a peak of the inspiration flow rate is detected after the detection of the start of the inspiration. When the respiration analyzing section 117 determines that a peak of the inspiration flow rate is detected, the respiration analyzing section 117 advances the processing to S311. When the respiration analyzing section 117 does not determine that a peak of the inspiration flow rate is detected, the respiration analyzing section 117 returns the processing to S305.

In S311, the respiration analyzing section 117 updates the predicted length of time that would be taken for the inspiration flow rate to reach a peak from the start of the inspiration.

In S313, the respiration analyzing section 117 determines whether the pressure reduction start timing is detected. When the respiration analyzing section 117 determines that the pressure reduction start timing is detected, the respiration analyzing section 117 notifies the treatment pressure control section 118 and advances the processing to S315. When the respiration analyzing section 117 does not determine that the pressure reduction start timing is detected, on the other hand, the respiration analyzing section 117 repeatedly performs S313. Incidentally, in a case where the supply air pressure has not reached the treatment pressure, the treatment pressure control section 118 continues the pressure raising control until the supply air pressure reaches the treatment pressure, in parallel with the respiration analyzing section 117 performing S311 and S313. When the supply air pressure has reached the treatment pressure, the treatment pressure control section 118 stops the pressure raising control and maintains the supply air pressure at the treatment pressure.

In S315, the treatment pressure control section 118 starts the pressure reducing control on the supply air pressure. That is, the treatment pressure control section 118 controls the blower 106 such that the supply air pressure decreases at a fixed rate (pressure reducing ratio) determined in advance in the program stored in the ROM 113. Incidentally, the treatment pressure control section 118 starts the pressure reducing control on the supply air pressure even when the supply air pressure has not reached the treatment pressure at the time of the execution of S315.

In S317, the treatment pressure control section 118 determines whether the supply air pressure has reached the lowest pressure. When the treatment pressure control section 118 determines that the supply air pressure has reached the lowest pressure, the treatment pressure control section 118 advances the processing to S319. When the treatment pressure control section 118 does not determine that the supply air pressure has reached the lowest pressure, the treatment pressure control section 118 repeatedly performs S317.

In S319, the treatment pressure control section 118 ends the pressure reducing control on the supply air pressure, and controls the blower 106 such that the supply air pressure maintains the lowest pressure. The treatment pressure control section 118 then returns the processing to S301.

As described above, according to the present embodiment, the supply air pressure is decreased before the sign of the measured value obtained by the flow sensor 105 changes from positive to negative (or the measured value obtained by the flow sensor 105 changes from a positive value to zero) and at a fixed pressure reducing ratio. It is thus possible to alleviate a difficulty in exhalation as compared with control that stops the blower 106 at a time point of change of the sign of the measured value from positive to negative (or change of the measured value from a positive value to zero). In addition, in a case where the lowest pressure is set higher than 0 cmH₂O, it is possible to suppress a decrease in the treatment effect and raise the supply air pressure smoothly at a time of the pressure raising control.

In addition, when a start of an inspiration is detected, the supply air pressure is increased in such a manner as to reach the treatment pressure at a timing when the flow rate during the inspiration is expected to reach a peak. Hence, a natural pressure increase synchronized with an inspiration motion of the patient can be realized, so that the patient is not readily given a feeling of strangeness.

### Other Embodiments

The present disclosure can also be implemented by processing in which a program for implementing one or more functions of the foregoing embodiment is supplied to a system or a device via a network or a storage medium, and one or more processors in a computer of the system or the device read and execute a program. In addition, the present disclosure can also be implemented by a circuit (for example, an ASIC) that implements the one or more functions.

### Summary of Embodiments

### (Item 1)

A medical device for supplying gas of positive pressure to an airway of a patient, the medical device including:
controller configured to control supply pressure of the gas;
the controller being configured to start pressure reducing control on the supply pressure when a decrease in a flow rate of the gas by a predetermined value from a peak flow rate in an inspiration period of the patient is detected.

According to this item, the supply air pressure is decreased before the inspiration period is ended. It is thus possible to alleviate a difficulty in exhalation.

### (Item 2)

The medical device according to item 1, in which,
in the pressure reducing control, the controller controls the supply pressure such that the supply pressure decreases at a predetermined pressure reducing ratio.

According to this item, the control of the supply pressure is not stopped, and the supply pressure is gradually decreased. It is thus possible to alleviate a difficulty in exhalation.

### (Item 3)

The medical device according to item 1 or 2, in which,
when the supply pressure reaches a predetermined lowest pressure higher than zero, the controller controls the supply pressure such that the supply pressure maintains the lowest pressure.

According to this item, a decrease in a treatment effect can be suppressed, and the supply air pressure can be raised smoothly at a time of pressure raising control.

### (Item 4)

The medical device according to any one of items 1 through 3, in which
the predetermined value is equal to or more than 25% but equal to or less than 90% of the peak flow rate.

According to this item, the pressure reducing control can be started at a more appropriate timing.

### (Item 5)

The medical device according to any one of items 1 through 3, in which
the predetermined value is equal to or more than 30% but equal to or less than 50% of the peak flow rate.

According to this item, the pressure reducing control can be started at an even more appropriate timing.

### (Item 6)

The medical device according to any one of items 1 through 5, in which,
in a case where there are a plurality of peak flow rates in the inspiration period of the patient, the controller starts the pressure reducing control when detecting a decrease by the predetermined value from a first peak flow rate after a time at which the peak flow rate is predicted to be reached from a start of the inspiration period.

According to this item, the pressure reducing control can be started at an appropriate timing even when there are a plurality of peak flow rates in the inspiration period of the patient.

### (Item 7)

The medical device according to any one of items 1 through 6, in which
the controller starts pressure raising control on the supply pressure when determining, on the basis of the flow rate of the gas, that the patient has started an inspiration, and,
in the pressure raising control, the controller controls the supply pressure such that the supply pressure reaches a predetermined treatment pressure at a time at which the peak flow rate is predicted to be reached in the inspiration period of the patient.

According to this item, the supply air pressure is increased in such a manner as to reach the treatment pressure at a timing when the flow rate during the inspiration is expected to reach a peak. Hence, a natural pressure increase synchronized with an inspiration motion of the patient can be realized, so that the patient is not readily given a feeling of strangeness.

### (Item 8)

A medical device for supplying gas of positive pressure to an airway of a patient, the medical device including:
controller configured to control supply pressure of the gas;
the controller being configured to start pressure raising control on the supply pressure when determining, on the basis of a flow rate of the gas, that the patient has started an inspiration, and
the controller being configured to, in the pressure raising control, control the supply pressure such that the supply pressure reaches a predetermined treatment pressure at a time at which a peak flow rate is predicted to be reached in an inspiration period of the patient.

According to this item, the supply air pressure is increased in such a manner as to reach the treatment pressure at a timing when the flow rate during the inspiration is expected to reach a peak. Therefore, a natural pressure increase synchronized with an inspiration motion of the patient can be realized, so that the patient is not readily given a feeling of strangeness.

### (Item 9)

The medical device according to item 7 or 8, in which
the predicted time is obtained on the basis of lengths of time taken to reach the peak flow rate from starts of inspirations in most recent predetermined number of times of respiration.

According to this item, the supply pressure can be controlled in such a manner as to reach the treatment pressure at an appropriate timing.

### (Item 10)

The medical device according to item 7 or 8, in which,
when there are a plurality of peak flow rates in the inspiration period of the patient, the predicted time is obtained on the basis of a length of time taken to reach a first peak flow rate from the start of the inspiration.

According to this item, the supply pressure can be controlled in such a manner as to reach the treatment pressure at an appropriate timing even when there are a plurality of peak flow rates in the inspiration period.

### (Item 11)

The medical device according to item 7 or 8, in which
the predicted time is an average value or a weighted average value of lengths of time taken to reach the peak flow rate from starts of inspirations in most recent plurality of times of respiration.

According to this item, the supply pressure can be controlled in such a manner as to reach the treatment pressure at a more appropriate timing.

### (Item 12)

The medical device according to item 11, in which
the most recent plurality of times are equal to or more than two times but equal to or less than eight times.

According to this item, the supply pressure can be controlled in such a manner as to reach the treatment pressure at an even more appropriate timing.

### (Item 13)

The medical device according to any one of items 1 through 12, in which
the medical device is an artificial respirator or a continuous positive airway pressure device.

### (Item 14)

A control method of a medical device for supplying gas of positive pressure to an airway of a patient, the control method including:
detecting a decrease in a flow rate of the gas by a predetermined value from a peak flow rate in an inspiration period of the patient; and
starting pressure reducing control on a supply pressure of the gas in response to the detection of the decrease in the flow rate of the gas by the predetermined value from the peak flow rate in the inspiration period of the patient.

According to this item, the supply air pressure is decreased before an end of the inspiration period. It is thus possible to alleviate a difficulty in exhalation.

### (Item 15)

A control method of a medical device for supplying gas of positive pressure to an airway of a patient, the control method including:
detecting a start of an inspiration by the patient on the basis of a flow rate of the gas; and
when detecting the start of the inspiration by the patient, controlling a supply pressure of the gas such that the supply pressure reaches a predetermined treatment pressure at a time when a peak flow rate is predicted to be reached in an inspiration period of the patient.

According to this item, the supply air pressure is increased in such a manner as to reach the treatment pressure at a timing when the flow rate during the inspiration is expected to reach a peak. Hence, a natural pressure increase synchronized with an inspiration motion of the patient can be realized, so that the patient is not readily given a feeling of strangeness.

### (Item 16)

A non-transitory storage medium storing a program for making a computer of a medical device for supplying gas of positive pressure to an airway of a patient function as the controller of the medical device according to any one of items 1 through 13.

According to this item, the program or the non-transitory storage medium storing the program for implementing the medical device having the above-described effects is provided.

The present disclosure is not limited to the contents of the foregoing embodiments, and is susceptible of various changes and modifications without departing from the spirit and scope of the disclosure. Hence, claims are attached to make the scope of the disclosure public.

What is claimed is:

1. A medical device for supplying gas of positive pressure to an airway of a patient, the medical device comprising:
controller configured to control supply pressure of the gas;
the controller being configured to start pressure reducing control on the supply pressure when a decrease in a flow rate of the gas by a predetermined value from a peak flow rate in an inspiration period of the patient is detected.

2. The medical device according to claim 1, wherein,
in the pressure reducing control, the controller controls the supply pressure such that the supply pressure decreases at a predetermined pressure reducing ratio.

3. The medical device according to claim 1, wherein,
when the supply pressure reaches a predetermined lowest pressure higher than zero, the controller controls the supply pressure such that the supply pressure maintains the lowest pressure.

4. The medical device according to claim 1, wherein
the predetermined value is equal to or more than 25% but equal to or less than 90% of the peak flow rate.

5. The medical device according to claim 1, wherein
the predetermined value is equal to or more than 30% but equal to or less than 50% of the peak flow rate.

6. The medical device according to claim 1, wherein,
in a case where there are a plurality of peak flow rates in the inspiration period of the patient, the controller starts the pressure reducing control when detecting a decrease by the predetermined value from a first peak flow rate after a time at which the peak flow rate is predicted to be reached from a start of the inspiration period.

7. The medical device according to claim 1, wherein
the controller starts pressure raising control on the supply pressure when determining, on a basis of the flow rate of the gas, that the patient has started an inspiration, and,
in the pressure raising control, the controller controls the supply pressure such that the supply pressure reaches a predetermined treatment pressure at a time at which the peak flow rate is predicted to be reached in the inspiration period of the patient.

8. A medical device for supplying gas of positive pressure to an airway of a patient, the medical device comprising:
controller configured to control supply pressure of the gas;
the controller being configured to start pressure raising control on the supply pressure when determining, on a basis of a flow rate of the gas, that the patient has started an inspiration, and
the controller being configured to, in the pressure raising control, control the supply pressure such that the supply pressure reaches a predetermined treatment pressure at a time at which a peak flow rate is predicted to be reached in an inspiration period of the patient.

9. The medical device according to claim 8, wherein
the predicted time is obtained on a basis of lengths of time taken to reach the peak flow rate from starts of inspirations in most recent predetermined number of times of respiration.

10. The medical device according to claim 8, wherein,
when there are a plurality of peak flow rates in the inspiration period of the patient, the predicted time is obtained on a basis of a length of time taken to reach a first peak flow rate from the start of the inspiration.

11. The medical device according to claim 8, wherein
the predicted time is an average value or a weighted average value of lengths of time taken to reach the peak flow rate from starts of inspirations in most recent plurality of times of respiration.

12. The medical device according to claim 11, wherein
the most recent plurality of times are equal to or more than two times but equal to or less than eight times.

13. The medical device according to claim 1, wherein
the medical device is an artificial respirator or a continuous positive airway pressure device.

14. A control method of a medical device for supplying gas of positive pressure to an airway of a patient, the control method comprising:
detecting a decrease in a flow rate of the gas by a predetermined value from a peak flow rate in an inspiration period of the patient; and
starting pressure reducing control on a supply pressure of the gas in response to the detection of the decrease in the flow rate of the gas by the predetermined value from the peak flow rate in the inspiration period of the patient.

15. A control method of a medical device for supplying gas of positive pressure to an airway of a patient, the control method including:
detecting a start of an inspiration by the patient on a basis of a flow rate of the gas; and
when detecting the start of the inspiration by the patient, controlling a supply pressure of the gas such that the supply pressure reaches a predetermined treatment pressure at a time when a peak flow rate is predicted to be reached in an inspiration period of the patient.

16. A non-transitory storage medium storing a program for making a computer of a medical device for supplying gas of positive pressure to an airway of a patient function as the controller of the medical device according to claim 1.
